(19) United States
(12) Patent Application Publication
(10) Pub. No.: US 2003/0032817 A1
(43) Pub. Date: Feb. 13, 2003

(54) Process for the synthesis of oxandrolone

(76) Inventors: John E. Cabaj, Sheboygan, WI (US); David L. Kairys, Sheboygan, WI (US); Paul M. Zizelman, Grafton, WI (US)

Correspondence Address: QUARLES & BRADY LLP, 411 E. WISCONSIN AVENUE, SUITE 2040, MILWAUKEE, WI, 53202-4497, US

(21) Appl. No.: 10/146,595

(22) Filed: May 15, 2002

Related U.S. Application Data
(60) Provisional application No. 60/290,966, filed on May 15, 2001.

(51) Int. Cl. C07D 311/78; C07J 1/00

(52) U.S. Cl. 549/276; 552/623

(57) ABSTRACT

A process is disclosed for synthesizing oxandrolone 1 involving the bromination of compound 2 to obtain compound 3, followed by the highly selective de-bromination of compound 3 to obtain compound 4, followed by the oxidation of compound 4 to obtain compound 6, and finally the reduction of compound 6 to obtain oxandrolone 1.

### CROSS-REFERENCE TO RELATED APPLICATIONS

This application claims priority to U.S. provisional application serial No. 60/290,966 filed May 15, 2001.

### STATEMENT REGARDING FEDERALLY SPONSORED RESEARCH OR DEVELOPMENT

Not applicable.

### BACKGROUND OF THE INVENTION

Oxandrolone (1) is an anabolic steroidal lactone currently being used to promote weight gain and for the relief of bone pain associated with osteoporosis. This invention is directed to a process for making oxandrolone which is efficient and easily scalable.

### SUMMARY OF INVENTION

We have discovered a process by which large quantities of oxandrolone can be produced. We found that the current literature methods to synthesize this compound are not suitable for large-scale production. One aspect of our new scaleable process is shown in FIG. 1.

### Bromination of Methylandrostanolone

One aspect of the present invention provides a process for making a compound of structure 3 comprising providing a compound of structure 2 in a reaction medium selected from the group consisting of an ethereal solvent, a chlorinated solvent, and acetonitrile; and brominating the compound of structure 2 with a source of electrophilic bromine to obtain the compound of structure 3. By ethereal we mean a liquid characterized by high volatility which often contains an ether. The source of electrophilic bromine may be selected from the group consisting of R₁R₂R₃R₄NBr₃, substituted or unsubstituted pyridinium tribromide, N-bromosuccinimide, 1,3-dibromo-5,5-dimethylhydantoin, and molecular bromine, wherein R₁ through R₄ are independently selected from alkyl or aryl groups. By substituted pyridinium tribromide we mean that the ring has subsituents which can include alkyl, aryl, halogens, and alkoxy groups. The alkyl group is preferably straight or branched, saturated or unsaturated having from 1 to 6 carbons. The aryl group may have 1 to 3 rings. The ethereal solvent may be selected from the group consisting of tetrahydrofuran, diethyl ether, 1,2-dimethoxyethane, 2-methoxy ethyl ether, and 1,4-dioxane. The chlorinated solvent may be selected from the group consisting of methylene chloride, chloroform, carbon tetrachloride, and 1,2-dichloroethane. Preferably, the brominating step is conducted at a temperature from −20° C. to room temperature and more preferably from 0° C. to 10° C. The process can include the further step of recovering the compound of structure 3.

Bromination of methylandrostanolone 2 (available from Schering-Plough and other suppliers) according to the procedure reported in U.S. Pat. No. 3,128,283 (Br₂, NaOAc, acetic acid) resulted in an extremely low yield of the desired bromide 3. However, we have discovered that bromination with phenyltrimethylammonium tribromide according to the procedure below gives 3 in 70-90% yield with acceptable purity. Thus, to a THF solution of 2 at 0° C. to 10° C. is added a THF solution of phenyltrimethylammonium tribromide over 2-3 hours. After stirring for an additional 1-2 hours the reaction is then quenched, preferably with an aqueous solution of sodium carbonate. The layers are then separated, the aqueous layer is extracted, preferably with ethyl acetate, and the combined organic extracts are concentrated to a low volume to give a thick slurry. The product is further precipitated, preferably with n-heptane, cooled, filtered and dried to constant weight to give 2-bromo-17-alpha-methyl-5-alpha-androstan-17-beta-ol-3-one (3) in 70-90%. While THF is the preferred solvent, the reaction can also be run in other ethereal solvents such as diethyl ether, chlorinated solvents such as methylene chloride and chloroform, and acetonitrile. Other nitrogen based tribromides can also be used as a source of electrophilic bromine. For example, under similar reaction conditions pyridinium tribromide gives 3 in 80% yield.

Bromination with molecular bromine in THF or diethyl ether at ca. 0° C. to 10° C. also affords 3; however, the purity is somewhat lower compared to bromination with tribromides. Bromination with N-bromosuccinimide as well as with 1,3-dibromo-5,5-dimethylhydantoin gives 3 but in somewhat lower yield and purity than with tribromides.

### Synthesis of Enone 4

Another aspect of the present invention provides a process of making a compound of structure 4 comprising providing a compound of structure 3 in an aprotic polar organic reaction medium; and debrominating the compound of structure 3 by adding a compound selected from the group consisting of lithium bromide, lithium fluoride, and magnesium bromide along with lithium carbonate to the reaction medium and heating the reaction medium to obtain the compound of structure 4. By aprotic we mean a moiety which neither donates nor accepts protons. The aprotic polar reaction medium may be selected from the group consisting of N,N-dimethylforamide, N,N-dimethylacetamide, 1-methyl-2-pyrrolidinone, and N,N′-dimethylpropylene urea. The process can include the further step of recovering the compound of structure 4.

Treatment of 3 with lithium chloride and lithium carbonate in refluxing DMF according to the U.S. Pat. No. 3,128,283 resulted in a low yield of the desired enone 4. In addition, the ratio of 4 to methyl testosterone 5 produced was 3/1 by HPLC analysis of the crude reaction product. We have found that replacement of lithium chloride with lithium bromide according to the procedure below surprisingly gives a 20/1 ratio of 4/5 by HPLC analysis of the crude reaction product. Thus, to a DMF slurry of 3 is added lithium carbonate and lithium bromide. The slurry is then heated at 110° C. to 115° C. for ca. 2-3 hours. Ethyl acetate is then added at room temperature followed by an aqueous solution of acetic acid. The layers are then separated, the aqueous layer is extracted with ethyl acetate, and the combined organic extracts are washed with water. The organic layer is then concentrated to a low volume to give a thick slurry. The product is further precipitated, preferably with n-heptane, cooled, filtered and dried to constant weight to give 17-beta -hydroxy-17-alpha-methyl-5-alpha-androst-1-ene-3-one (4) in 60-75% from 3. Lithium fluoride or magnesium bromide can be used in place of LiBr but the ratio of 4/5 is lowered to 8/1 and 10/1 respectively. Other polar aprotic solvents such as N,N-dimethylacetamide can also be used.

### Oxidation of 4 to 6

Yet another aspect of the present invention provides a process for making a compound of structure 6 comprising providing a compound of structure 4 in a lower alkanol reaction medium; oxidizing the compound of structure 4 with ozone to form an initial ozonolysis adduct; converting the initial ozonolysis adduct to a salt of the compound of structure 6 by adding a base to the reaction medium; and acidifying the salt to obtain the compound of structure 6. By lower alkanol we mean a straight or branched alcohol having from 1 to 6 carbons. The lower alkanol reaction medium is preferably methanol. The base can be any alkali metal or alkaline earth metal base but is preferably aqueous sodium hydroxide. The acidifying agent can be any mineral acid but is preferably HCl. The process can include the further step of recovering the compound of structure 6.

U.S. Pat. No. 3,128,283 describes the oxidation of 4 to 6 using osmium tetroxide in the presence of lead tetraacetate. The extreme toxicity of both of these reagents makes this method clearly undesirable for large-scale production. With this concern in mind, we developed an ozonolysis process for achieving this conversion. U.S. Pat. No. 3,109,016 describes the ozonolysis of 4 in carbon tetrachloride to give the formic acid mixed anhydride of 6 (compound 7) after stirring the intermediate ozonide in methylene chloride.

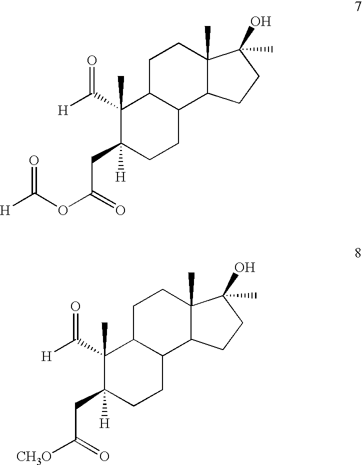

Alternatively, the methyl ester of 6 (compound 8) is obtained when the ozonolysis is performed in methanol. In both of these procedures, the potentially dangerous ozonide or peroxide intermediates are presumably decomposed thermally. The use of carcinogenic carbon tetrachloride as the solvent is not suitable for large-scale production. Our method for performing this oxidation calls for ozonation of 4 at −30 to −40° C., preferably in methanol. After the reaction is judged to be complete, the initial ozonolysis adduct is converted to the sodium salt of 6, preferably by the addition of aqueous sodium hydroxide at −10° C. Methanol is then removed and the resulting aqueous solution of the sodium salt of 6, preferably is washed with t-butyl methyl ether. The pH of the aqueous layer is then adjusted to ca. 4, preferably with aqueous HCl, the resulting slurry is then filtered, washed with water preferably followed by n-heptane, and finally dried to give 17-beta-hydroxy-17-alpha-methyl-1-oxo-1,2-seco-A-nor-5-alpha -androstan-2-oic acid 6 in 75-85% yield. The crude product is then recrystallized, preferably from methanol and water, to give recrystallized 6 in 85-95% yield.

### Synthesis of Oxandrolone from 6

The aldehyde 6 was reduced to the seco-acid of oxandrolone via the addition of NaBH₄ to an ethanol/water solution of the sodium salt of 6 at 0-5° C. Upon reaction completion, the pH of the reaction mixture is carefully adjusted to 1-2, preferably with hydrochloric acid. The slurry is stirred for ca. 3 hours at 0-10° C. to effect cyclization of the intermediate seco-acid. The slurry of crude oxandrolone is then filtered, washed, and dried to give oxandrolone (1) in 85-95% yield.

A still further aspect of the present invention provides a process for making a compound of structure 1 comprising providing a compound of structure 2 in a reaction medium selected from the group consisting of an ethereal solvent, a chlorinated solvent, and acetonitrile; brominating the compound of structure 2 with a source of electrophilic bromine to obtain a compound of structure 3; providing the compound of structure 3 in an aprotic polar organic reaction medium; debrominating the compound of structure 3 by adding a compound selected from the group consisting of lithium bromide, lithium fluoride, magnesium bromide, and lithium perchlorate along with lithium carbonate to the reaction medium and heating the reaction medium to obtain a compound of structure 4; providing the compound of structure 4 in a lower alkanol reaction medium; oxidizing the compound of structure 4 with ozone to form an initial ozonolysis adduct; converting the initial oxonolysis adduct to a salt of a compound of structure 6 by adding a base to the reaction medium; acidifying the salt to obtain the compound of structure 6; providing the compound of structure 6 in an aqueous alcohol reaction medium; reducing the compound of structure 6 to the seco-acid of oxandrolone; and stirring the reaction medium of the seco-acid of oxandrolone to cyclize the seco-acid to obtain the compound of structure 1.

### BRIEF DESCRIPTION OF THE DRAWING

FIG. 1 is a schematic depicting the process according to one aspect of the invention (Me=CH₃).

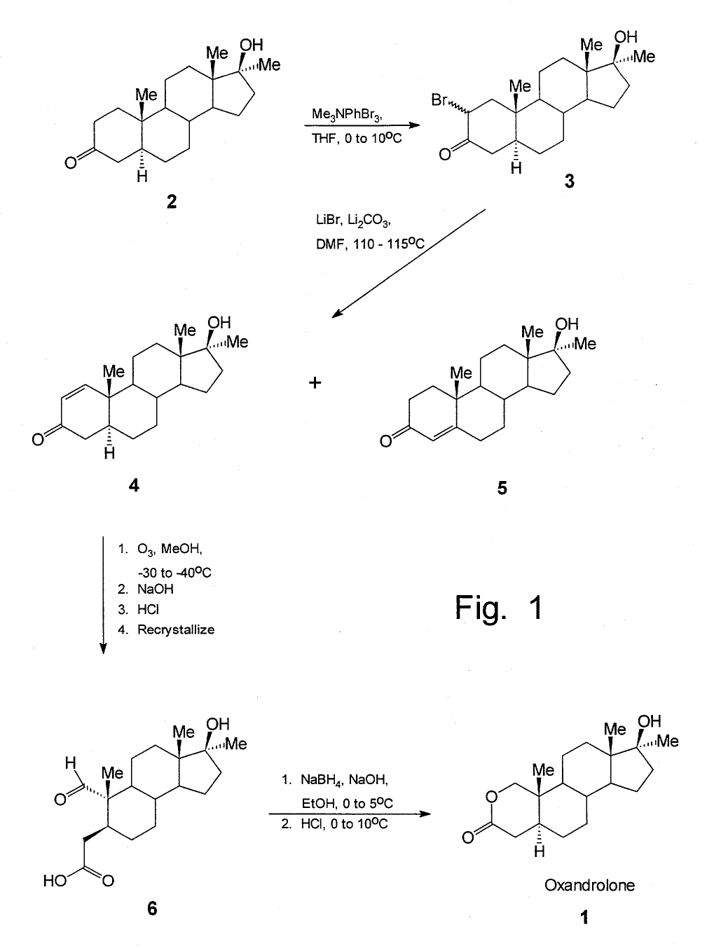

### DETAILED DESCRIPTION OF THE INVENTION

### Example 1

### Preparation of 2-Bromo-17-alpha-methyl-5-alpha -androstan-17-beta-ol-3one (3) using Phenyltrimethylammonium Tribromide

To a nitrogen purged 50-L 4 neck round bottom flask equipped with an addition funnel, temperature probe, nitrogen inlet adapter, and stirrer apparatus, was added 650 g (2.14 mol) of methylandrostanolone (2) and 5.8 L of THF. The solution was cooled to an internal temperature of 0° C. to 10° C.

To the solution was added a THF solution of phenyltrimethylammonium tribromide (884 g, 2.36 mol, 2-L of THF) over ca. 1 hour while maintaining an internal temperature of 0° C. to 10° C. When the addition was complete the resulting thick slurry was stirred for ca. 1 hour at 0° C. to 10° C. at which point TLC analysis indicated reaction completion. To the slurry was added an aqueous solution of Na₂CO₃ (377 g in 3.9 L of water) over ca. 1 hour at 0° C. to 10° C. Four liters of additional water were added and the mixture was allowed to warm to room temperature. The layers were then allowed to settle for ca. 1 hour and then separated. The aqueous layer was extracted twice with 4 L of ethyl acetate and the combined organic extracts were washed with 4.5 L of water. The organic layer was then concentrated using vacuum to a volume of ca. 2 L. To the resulting white slurry at ca. room temperature was added 4.6 L of n-heptane over 2 hours. The slurry was cooled to ca. 0° C. to 10° C. and held at this temperature for ca. 2 hours. The slurry was then filtered through a course porosity fritted funnel, washed three times with 600 mL of 0° C. to 10° C. n-heptane, and dried to a constant weight under high vacuum at ca. 40° C. to give 621 g (76% yield) of 2-bromo-17-alpha-methyl -5-alpha-androstan-17-beta-ol-3-one (3) as a pink solid (mp. 195-197° C. (uncorrected)).

### Example 2

### Preparation of 3 using Phenyltrimethylammonium Tribromide in Methylene Chloride

To a 0° C. to 5° C. solution of 2 in CH₂Cl₂ (0.50 g, 1.6 mmol, in 5.0 mL of CH₂Cl₂) was added 0.68 g (1.8 mmol) of phenyltrimethylammonium tribromide in 10 mL of CH₂Cl₂. After stirring for 3 hours at 0° C. to 5° C., 2.0 mL of a saturated solution of sodium metabisulfite was added at 0° C. to 5° C. Five milliliters of saturated sodium bicarbonate was then added. The layers were allowed to separate, the aqueous layer was extracted with 5 mL of CH₂Cl₂, and the combined organic extracts were washed with 5 mL of water. The organic layer was then dried over Na₂SO₄, concentrated, and dried via high vacuum at room temperature to give 0.57 g of 3 (93% yield) as a tan solid.

### Example 3

### Preparation of 3 using Phenyltrimethylammonium Tribromide in Diethyl Ether

To a 0° C. to 10° C. slurry of 2 in diethyl ether (0.50 g, 1.6 mmol, 10 mL of diethyl ether) was added a slurry of 0.68 g (1.8 mmol) of phenyltrimethylammonium tribromide in 3 mL of diethyl ether. The reaction was stirred for 3 hours at which point 8 mL of additional diethyl ether was added to improve stirring. The reaction was stirred for an additional 2 hours. Eight milliliters of saturated NaHCO₃ was then added over ca. 5 minutes at 0 to 10° C. Thirty milliliters of additional water was added and the slurry was allowed to warm to room temperature. The layers were then separated and the aqueous layer, which contained some solid material, was extracted twice with 10 mL of ethyl acetate. The combined organic extracts were washed with 20 mL of water, dried over Na₂SO₄, concentrated, and dried via high vacuum to give 0.35 g of 3 (64%) as a tan solid.

### Example 4

### Preparation of 3 using in Phenyltrimethylammonium Tribromide in Chloroform

To a 0° C. to 10° C. solution of 2 in CHCl₃ (0.50 g, 1.6 mmol, in 5.0 mL of CHCl₃) was added a slurry of 0.68 g (1.8 mmol) of phenyltrimethylammonium tribromide in 10 mL of CHCl₃

After stirring for 1 hour at 0° C. to 10° C., 3.0 mL of a saturated solution of sodium bicarbonate was added at 0° C. to 5° C. The layers were allowed to separate, the aqueous layer was extracted twice with 7 mL of CHCl₃, and the combined organic extracts were washed with 10 mL of water. The organic layer was then dried over Na₂SO₄, concentrated, and dried via high vacuum at room temperature to give 0.23 g of 3 (36% yield) as a tan solid.

### Example 5

### Preparation of 3 using Pyridinium Tribromide

To a 0° C. to 5° C. solution of 2 in THF (0.50 g, 1.6 mmol, in 5.0 mL of THF) was added 0.51 g (1.6 mmol) of pyridinium tribromide in 4.0 mL of THF. After stirring for 3 hours at 0° C to 10° C., 2.0 mL of a saturated solution of sodium metabisulfite was added at 0° C. to 10° C. Five milliliters of saturated sodium bicarbonate was then added. The reaction mixture was then allowed to warm to room temperature. Twenty-five milliliters of ethyl acetate and 25 mL of water were then added and the biphasic slurry was filtered through celite. The celite was washed thoroughly with ethyl acetate. The filtrate layers are separated and the aqueous phase was extracted with 20 mL of ethyl acetate. The combined organic extracts were dried over Na₂SO₄, concentrated, and finally dried on a high vacuum at room temperature to give 0.49 g of 3 (80% yield) as a white solid.

### Example 6

### Preparation of 3 using Bromine in THF

To a 6° C. suspension of 2 in THF (0.50 g, 1.6 mmol, in 5.0 mL of THF) was added 88 microliters of bromine in 2.0 mL of THF over 5 minutes. After 1.5 hours of stirring at 0° C. to 10° C., 3.0 mL of saturated NaHCO₃ was added to the white slurry. Five milliliters of ethyl acetate and 2.0 mL of water were then added to the quenched reaction. The layers were then separated and the aqueous layer was extracted with 5.0 mL of ethyl acetate. The combined organic layers were washed with 5.0 mL of water, dried over Na₂SO₄ and concentrated to a solid. The solid was then dried on a high vacuum at room temperature to give 0.57 g of 3 (93%).

### Example 7

### Preparation of 17-beta-hydroxy-17-alpha-methyl-5-alpha-androst-1-ene-3-one (4) using LiBr, Li₂CO₃

To a 5-liter four neck round bottom flask equipped with a thermoprobe, nitrogen inlet adapter, and mechanical stirrer was added 200 g (0.52 mol) of 3, and 1.2 L of DMF. To the slurry at room temperature was added 42.6 g (0.58 mol) of Li₂CO₃ followed by 76.2 g (0.89 mol) of LiBr. There was an approximate 10° C. exotherm upon addition of LiBr. The slurry was then heated to 110° C. to 115° C. and held at this temperature for 3 hours at which point TLC analysis indicated reaction completion. The slurry was cooled to an internal temperature of 350° C. and 1.4 L of ethyl acetate was added. An aqueous solution of acetic acid (62 mL acetic acid in 600 mL of water) was then added over ca. 0.5 hours. The reaction mixture was then stirred at room temperature for ca. 8 hours and then held at 0° C. to 10° C. for 2 days. The reaction mixture was then warmed to room temperature, the layers were separated, and the aqueous layer was extracted three times with 1.2 L of ethyl acetate. The combined organic extracts were washed three times with 1.0 L of water to remove the majority of the DMF and concentrated via vacuum to a total volume of ca. 500 mL. To the resulting slurry was added 1.8 L of n-heptane over 0.5 hours at room temperature. The slurry was cooled to an internal temperature of 0° C. to 10° C. and held at this temperature for ca. 2 hours. The slurry was then filtered, washed twice with 200 mL of 0° C. to 10° C. n-heptane, and dried to constant weight via high vacuum at ca. 45° C. overnight to give 128 g (81% yield) of 17-beta-hydroxy-17-alpha-methyl-5-alpha -androst-1-ene-3-one (4) as an off-white solid (mp. 153° C. -154° C. (uncorrected))

### Example 8

### Preparation of 4 using LiBr, Li₂CO₃ in N,N-Dimethylacetamide

To 5.00 g (13.1 mmol) of 3 in 38 mL of N,N-dimethylacetamide was added 1.95 g (22.4 mmol) of LiBr and 1.06 g (14.4 mmol) of Li₂CO₃. The slurry was then heated at 84° C. to 105° C. for 6 hours. The slurry was cooled to 8° C. and the pH was adjusted to 4 with acetic acid. Thirty-eight milliliters of water was then added dropwise. The resulting slurry was stirred at 0° C. to 5° C. for 1 hour, filtered, washed with 34 mL of water, and dried via high vacuum at 50° C. to give 3.89 g of 4 (97%).

### Example 9

### Preparation of 4 using LiF, Li₂CO₃

Five grams (13 mmol) of 3 in 41 mL of DMF was treated with 0.41 g (16 mmol) of LiF and 1.06 g (14.4 mmol) of Li₂CO₃. The resulting slurry was then heated at 90° C. to 93° C. for 3 hours. An additional 0.17 g (6.6 mmol) of LiF was then added and the reaction was heated for an additional 6.5 hours at 90° C. to 950° C. The slurry was then cooled to 0° C. to 5° C. One milliliter of acetic acid was then added followed by 35 mL of water. The slurry was stirred for 45 minutes, filtered, washed with 15 mL of water, and dried via high vacuum at room temperature to give 3.8 g of 4 (96% recovery, 4/5=8/1 by area percent HPLC).

### Example 10

### Preparation of 4 using MgBr₂, Li₂CO₃

To 5.00 g (13.1 mmol) of 3 in 40 mL of DMF was added 1.06 g (14.4 mmol) of Li₂CO₃ and 7.26 g (39.5 mmol) of MgBr₂. The slurry was heated at 100° C. to 110° C. for 7 hours. The slurry was cooled to 0° C. to 5° C. Thirty-five milliliters of 1M HCl was then added dropwise. The slurry was stirred at 0° C. to 5° C. for 1 hour, filtered, washed thoroughly with water, and dried on a high vacuum at 40° C. to give 3.59 g of 4 (91%, 4/5=10/1 by area percent HPLC).

### Example 11

### Preparation of 17-beta-hydroxy-17-alpha-methyl-1-oxo-1,2-seco-A-nor-5-alpha-androstan-2-oic acid (6)

Two hundred fifty grams (0.828 moles) of 4 was dissolved in 2.5 L of MeOH and cooled to −30° C. to −40° C. Ozone was then bubbled into the solution through a sparge tube. After 6 hours the reaction was judged to be complete by TLC analysis. The solution was allowed to warm to −10° C. and an aqueous solution of NaOH (109 mL concentrated NaOH in 3.13 L H₂O) was added dropwise over 2 hours, with an exotherm of 8° C. Once at room temperature, the mixture was concentrated under reduced pressure, with a total of approximately 2.8 L of MeOH being removed. The aqueous solution of the sodium salt was washed twice with 1 L of t-butyl methyl ether, and acidified with concentrated HCl (120 mL) to a pH of 4. The resulting slurry was stirred for about 1 hour after completion of the acid addition, then filtered, washed twice with 300 mL of H₂O followed by 200 mL of n-heptane. The solid product was then dried under high vacuum to a constant weight at 40 ° C. to 60° C. to give 210.9 g of 6 (79% yield).

### Example 12

### Recrystallization of 6

To a 50-L four neck round-bottom flask equipped with a stir assembly, condenser, thermocouple, nitrogen inlet adapter, 2-L addition funnel, and heating mantle, were charged 2.96 kg of 6. To the solids were added 20.7 L of methanol. The solution of 6 was heated to an internal temperature of 59° C. To the solution was added 25.7 L of water over a 2 hour period at 59° C. to 65° C. When the water addition was complete the resulting slurry was cooled slowly to room temperature by leaving the flask in the heating mantle. The slurry was then further cooled to 0° C. to 10° C. and held at this temperature for ca. 2 hours. The slurry was filtered, washed with 1 L of 0° C. to 5° C. 2/1 water/methanol (by volume), followed by portion-wise washing of the cake with an additional 6 L of the cold 2/1 mixture. The highly crystalline white solid was then dried at 45° C. in a high vacuum to constant weight to give 2.11 kg (71% yield) of recrystallized 6 (mp =176° C. - 179° C. (uncorrected)).

### Example 13

### Preparation of Oxandrolone (1, 17-beta-hydroxy-17-alpha-methyl-2-oxa-5-alpha-androstane-3-one)

Two hundred six grams (0.64 moles) of 6 was slurried in 1.54 L of ethanol and 1.54 L H₂O. The slurry was cooled to 0-10° C. using ice water. Sodium hydroxide (74 mL of a 9.5 M aqueous solution, 0.70 moles) was added dropwise over ten minutes. To the resulting solution was added 36.29 g (0.96 moles) of NaBH₄ in portions over 1.5 hours. After stirring for an additional 1 hour after the completion of the addition, the reaction was judged to be complete by TLC analysis. The pH of the solution was carefully adjusted to 1 to 2 by the addition of 6 M aqueous HCl. The resulting slurry was allowed to stir for an additional 3 hours at which point the cyclization reaction was judged to be complete by TLC analysis. The white slurry was then filtered, washed with 250 mL of 0° C. to 5° C. 1/1 EtOH/H₂O (by volume), 600 mL H₂O, and 700 mL of n-heptane. The solids were dried via high vacuum to constant weight at 40-60° C. to give 168.69 g of 1 (86% yield)

We claim:

1. A process for making a compound of structure 3, comprising:
providing a compound of structure 2 in a reaction medium selected from the group consisting of an ethereal solvent, a chlorinated solvent, and acetonitrile; and
brominating the compound of structure 2 with a source of electrophilic bromine to obtain the compound of structure 3.

2. The process of claim 1, wherein the source of electrophilic bromine is selected from the group consisting of. R₁R₂R₃R₄NBr₃, substituted or unsubstituted pyridinium tribromide, N-bromosuccinimide, 1,3-dibromo-5,5-dimethylhydantoin, and molecular bromine, wherein R₁ through R₄ are independently selected from alkyl or aryl groups.

3. The process of claim 1, wherein the ethereal solvent is selected from the group consisting of tetrahydrofuran, diethyl ether, 1,2-dimethoxyethane, 2-methoxy ethyl ether, and 1,4-dioxane.

4. The process of claim 1, wherein the chlorinated solvent is selected from the group consisting of methylene chloride, chloroform, carbon tetrachloride, and 1,2-dichloroethane.

5. The process of claim 1, wherein the brominating step is conducted at a temperature from −20° C. to room temperature.

6. The process of claim 5, wherein the temperature is from 0° C. to 10° C.

7. The process of claim 1, comprising the further step of recovering the compound of structure 3.

8. A process of making a compound of structure 4, comprising:
providing a compound of structure 3 in an aprotic polar organic reaction medium; and
debrominating the compound of structure 3 by adding a compound selected from the group consisting of lithium bromide, lithium fluoride, and magnesium bromide along with lithium carbonate to the reaction medium and heating the reaction medium to obtain the compound of structure 4.

9. The process of claim 8, wherein the aprotic polar reaction medium is selected from the group consisting of N,N-dimethylforamide, N,N-dimethylacetamide, 1-methyl-2-pyrrolidinone, and N,N′-dimethylpropylene urea.

10. The process of claim 8, comprising the further step of recovering the compound of structure 4.

11. A process for making a compound of structure 6, comprising:
providing a compound of structure 4 in a lower alkanol reaction medium;
oxidizing the compound of structure 4 with ozone to form an initial ozonolysis adduct;
converting the initial ozonolysis adduct to a salt of the compound of structure 6 by adding a base to the reaction medium; and
acidifying the salt to obtain the compound of structure 6.

12. The process of claim 11, wherein the lower alkanol reaction medium is methanol.

13. The process of claim 11, wherein the base is aqueous sodium hydroxide.

14. The process of claim 11, wherein the acidifying agent is HCl.

15. The process of claim 11, comprising the further step of recovering the compound of structure 6.

16. A process for making a compound of structure 1, comprising:
providing a compound of structure 2 in a reaction medium selected from the group consisting of an ethereal solvent, a chlorinated solvent, and acetonitrile;
brominating the compound of structure 2 with a source of electrophilic bromine to obtain a compound of structure 3;
providing the compound of structure 3 in an aprotic polar organic reaction medium;
debrominating the compound of structure 3 by adding a compound selected from the group consisting of lithium bromide, lithium fluoride, magnesium bromide, and lithium perchlorate along with lithium carbonate to the reaction medium and heating the reaction medium to obtain a compound of a structure 4;
providing the compound of structure 4 in a lower alkanol reaction medium;
oxidizing the compound of structure 4 with ozone to form an initial ozonolysis adduct;
converting the initial oxonolysis adduct to a salt of a compound of structure 6 by adding a base to the reaction medium;
acidifying the salt to obtain the compound of structure 6;
providing the compound of structure 6 in an aqueous alcohol reaction medium;
reducing the compound of structure 6 to the seco-acid of oxandrolone; and
stirring the reaction medium of the seco-acid of oxandrolone to cyclize the seco-acid to obtain the compound of structure 1.
